(19) United States
(12) Patent Application Publication
(10) Pub. No.: US 2019/0145985 A1
(43) Pub. Date: May 16, 2019

(54) METHOD FOR ELIMINATING TARGET INTERFERENCE

(71) Applicant: BioAgilytix Labs, LLC, Durham, NC (US)

(72) Inventors: Jennifer Mitchell Zemo, Fuquay Varina, NC (US); Carla Veronica Mejia, Raleigh, NC (US); Christina Nelson Strom, Durham, NC (US)

(21) Appl. No.: 16/177,658

(22) Filed: Nov. 1, 2018

Related U.S. Application Data
(60) Provisional application No. 62/584,312, filed on Nov. 10, 2017.

(51) Int. Cl. G01N 33/68

(52) U.S. Cl. CPC G01N 33/6854

(57) ABSTRACT

Described herein are methods and kits for eliminating or reducing drug target interference and improving drug tolerance in anti-drug antibody (ADA), pharmacokinetic, biomarker, or toxicological assays. The method comprises heating the biological assay sample to reduce target binding to the drug.

### CROSS REFERENCE TO RELATED APPLICATIONS

This application claims priority to U.S. Provisional Patent Application No. 62/584,312, filed Nov. 10, 2017, which is incorporated herein in its entirety by express reference thereto.

### TECHNICAL FIELD

Described herein is a method for eliminating or reducing drug target interference and improving drug tolerance during immunological assays such as anti-drug antibody (ADA), pharmacokinetic, biomarker, or toxicological assays.

### BACKGROUND

Biologic drugs, such as therapeutic proteins, are capable of inducing an immune response in the subject upon administration. The immune response can lead to the production of anti-drug antibodies (“ADAs”) that bind to the biologic and reduce its effectiveness and lead to pernicious side effects such as allergic reactions, cross-reactivity, and complement activation. Biologic drugs must be evaluated for immunogenicity during clinical development. Typically, a sample of blood, plasma, serum, or urine is screened for the presence of antibodies that react with the biological drug. These include both ADAs and neutralizing antibodies (NABs). It is important that ADA screening assays are valid, sensitive, specific, and selective for determining ADA responses to a given therapeutic protein. Screening assays represent a key aspect of therapeutic protein product development.

Anti-drug antibodies (ADAs) and neutralizing antibodies (NABs) can adversely affect the safety and efficacy of protein therapeutics. Knowing the extent of ADA and NAB production following administration of a protein therapeutic is critical. The commonly used ADA method is the bridging assay where a multi-valent ADA bridges between a capture drug (unlabeled or biotin labeled) and a labeled detection drug. The accuracy of ADA and NAB tests is influenced by numerous factors, which include circulating drug, endogenous drug homologs, drug targets, and serum factors. Of these, circulating drug interference (leading to a false negative) and drug target (leading to false positives) present a major challenge in developing an accurate ADA and NAB assay.

Circulating drug interference presents a problem because during the assay, excess circulating drug will complex with the ADA and the ADA will go undetected leading to erroneous false negative results. The maximal amount of free drug in a sample that still results in a detectable ADA signal is known as drug tolerance. The acid treatment or basic treatment of samples has been used to improve free drug tolerance in ADA assays. Following this treatment, antibody-antigen (or drug) binding is weakened and eventually disrupted by a low pH, in the case of acid treatment, or a high pH, in the case of basic treatment. This treatment makes the detection of free ADA that is dissociated from partially or completely drug-bound ADAs possible in many immunogenic assay formats (i.e., bridging assay formats), thereby improving drug tolerance. The structural characteristics of the biologic drug, such as pI, or the presence of certain conjugating bonds, will dictate whether an acid solution or basic solution is more appropriate for disrupting ADA/drug binding.

Other approaches developed to improve drug tolerance include the affinity capture elution (ACE). See Bourdage et al., J. Immunol. Methods 327(1-2):10-17 (2007). This method includes binding of an ADA from an acid treated sample to an immobilized drug followed by a second acid treatment step of the immobilized complex where only the ADA is released and subsequently detected. Another similar approach is the biotin-drug extraction with acid dissociation assay (BEAD), wherein the ADAs are captured on a drug coated bead and subsequently detected. See Lofgren et al., J. Immunol. Methods 308(1-2):101-108 (2006) and Xu et al., J. Immunol. Methods 416:94-104 (2015). A similarly employed technique is the solid-phase extraction with acid dissociation (SPEAR), which uses a biotin-avidin to capture the ADA/drug complexes. Other methods include adding excess-labeled drug during drug detection to outcompete the drug present in the sample. Another method utilizes precipitation and acid dissociation (PanDA), where excess drug is used to complex all ADAs and the complexes are precipitated with polyethylene glycol (PEG), disassociated, and the amount of ADA is detected. See Zoghbi et al., J. Immunol. Methods 426:62-69 (2015) and U.S. Pat. No. 9,759,732, which are incorporated by reference herein for the specific teachings thereof.

Thus, while many drug-tolerance assays have been contemplated, accounting for drug-target interference remains a major hurdle in the field. For example, excess drug target present in the serum can affect the read-out of the amount of ADA in a sample and lead to false positive results in commonly used bridging ADA assay formats or false negatives in other assay formats. In addition, the presence of drug target can lead to intra and inter person variability even at various sampling time points owing to fluctuations in the amount of drug target present in the source (e.g., serum) from which a biological sample is taken. As mentioned above, acid treatment to disassociate drug/ADA complexes is commonly used in the aforementioned approaches to improve drug tolerance. However, this same treatment can exacerbate drug target interference through also disassociating drugs from their target. This step effectively provides additional target in the sample that can increase target interference, which is particularly problematic for those targets that can multimerize.

Alternatively, methods to improve target interference often employ target binding or neutralization. It has been contemplated to use anti-target antibodies, which are specific to the drug target to separate or neutralize the drug target. However, an anti-target antibody with sufficiently high specificity is required, which is not always available or would be lengthy and complex to generate. Complex validation is required to ensure that the antibody does not remove any of the ADA. In addition, in the case where the biotherapeutic neutralizes a receptor ligand, the use of soluble receptors can be employed, which bind to the ligands and reduce target interference. Often receptors are not soluble (as in the case of membrane bound receptors) or recombinant versions may be conformationally different, and there is a high cost associated with purifying and characterizing these receptors for each ADA assay in addition to complex validation steps. Lastly, PEG precipitation may be useful in increasing drug tolerance and reducing target interference; however, this method is tedious and time consuming, and not particularly well suited for the analysis of large numbers of samples, which is often the case in clinical trials. Thus, there is an unmet need for a simple, yet effective approach for reducing drug target interference and improving drug tolerance in immunogenicity assays.

### SUMMARY

Described herein is a method for detecting ADAs in a sample while reducing target interference and improving drug tolerance in an anti-drug antibody or NABs, and methods for determining whether a sample is positive or negative for an ADA or NAB. It was surprisingly and unexpectedly discovered that heating a sample suspected of having an anti-drug antibody for a time period was sufficient to reduce or eliminate target interference and improve drug tolerance in an anti-drug antibody detection assay. It was found that this discovery increases the specificity, selectivity, and reliability of an anti-drug antibody assay by reducing drug target interference. This methodology can also be used for pharmacokinetic, toxicological, and biomarker assays.

One embodiment described herein is a method for detecting anti-drug antibodies that are antigenic to a drug in a sample comprising: (a) obtaining a sample suspected to have one or more anti-drug antibodies; (b) coating a first substrate with the drug to create an immobilized drug coated substrate; (c) heating the sample for a time period, wherein the heating of the sample reduces drug target binding to the drug; (d) contacting the sample of step (c) with the first drug coated substrate of step (b) to form an immobilized complex between the drug coated on the substrate and the anti-drug antibody present in the sample; and (e) detecting the presence of anti-drug antibodies, if present, with a detection reagent. In another aspect, the sample is cooled following the heating step (c). In another aspect, the sample is further diluted in an antibody blocking buffer following the heating step (c). In another aspect, the sample is diluted to the minimum required dilution, wherein the minimal required dilution is a dilution of the sample, which yields a detection signal that is close to that of the diluent. In another aspect, the antibody blocking buffer comprises bovine serum albumin, mammalian sera (e.g., human, bovine, calf, horse, goat, rabbit, or mouse), casein, dried milk, commercial blocking agents, or a combination thereof. In another aspect, the sample is treated with an acid or a base for a time period, wherein the acid treatment disrupts binding of an anti-drug antibody to a drug prior to the contacting step (d). In another aspect, the acid comprises glycine, citrate, maleate, formate, fumarate, acetate, phosphate, carbonate, or HCl or combinations thereof. In another aspect, the acid comprises glycine at a concentration of about 0.1 M to about 1 M. In another aspect, the base comprises NaOH, KOH, NH₄OH, tris(hydroxymethyl)aminomethane (Tris base), trimethylamine, or bicarbonate salts or combinations thereof. In another aspect, the drug coated substrate is washed and a neutralizing agent is added to the substrate prior to the contacting step (d). In another aspect, the neutralizing agent comprises an acidic buffer or a basic buffer. In another aspect, the neutralizing buffer is a basic buffer having a pH of about 8 to about 11. In another aspect, the anti-drug antibody is disassociated from the immobilized complex on the first substrate and immobilized on a second substrate. In another aspect, the disassociation of the immobilized complex comprises further treating the immobilized complex on the first substrate with an acid or a base for a time period, wherein the acid treatment disrupts binding of the anti-drug antibody to the immobilized drug. In another aspect, the drug remains immobilized upon the first substrate. In another aspect, the acid comprises glycine at a concentration of about 0.1 M to about 1 M. In another aspect, the base comprises NaOH, KOH, NH₄OH, tris(hydroxymethyl)aminomethane (Tris base), trimethylamine, or bicarbonate salts or combinations thereof. In another aspect, the detection reagent comprises the drug conjugated to a detectable label. In another aspect, the detection reagent comprises a modification of the drug conjugated to a detectable label. In another aspect, the detectable label comprises an electrochemiluminescent label, chemiluminescent label, fluorescent, or an enzyme label. In another aspect, the modification of the drug comprises pegylation or glycosylation. In another aspect, the method further comprises titering the anti-drug antibody comprising progressively diluting the sample until the detection falls below the cut point. In another aspect, the sample is heated to a temperature within a range comprising: about 40° C. to about 100° C., about 50° C. to about 95° C., about 60° C. to about 95° C., about 60° C. to about 85° C., or about 60° C. to about 75° C. In another aspect, the sample is heated for a time period comprising: about 1 second, about 5 seconds, about 10 seconds, about 15 seconds, about 20 seconds, about 40 seconds, about 60 seconds, about 1 minute, about 2 minutes, about 5 minutes, about 10 minutes, about 20 minutes, about 30 minutes, or about 1 hour. In another aspect, the first substrate is coated with an excess of the drug compared to an amount of the drug present in the sample. In another aspect, the drug comprises a protein or a nucleic acid. In another aspect, the drug comprises an antibody or a peptide. In another aspect, the target is a cellular protein. In another aspect, the heating step denatures the target. In another aspect, the anti-drug antibody has an immunoglobulin isotype comprising IgG, IgA, IgM, IgE, or combinations thereof.

Another embodiment described herein is a method for determining whether a sample is positive or negative for having immunogenic anti-drug antibodies comprising performing the method described herein, wherein the sample is positive for an anti-drug antibody if it is above a pre-determined cut point. In one aspect, the method further comprises confirming the presence of the anti-drug antibody by adding an amount of unlabeled drug in the detection step, wherein the detection signal is reduced following the addition of unlabeled drug.

Another embodiment described herein is a method for detecting anti-drug antibodies that are antigenic to a drug in a sample comprising: (a) obtaining a sample suspected to have one or more anti-drug antibodies; (b) coating a first substrate with the drug to create an immobilized drug coated substrate; (c) heating the sample, wherein the heating step reduces drug target binding to the drug; (d) cooling the sample of step (c); (e) diluting the sample of step (d) in an antibody blocking buffer; (f) treating the sample of step (e) with an acid or a base to disassociate any drug and anti-drug antibodies to form a solution of disassociated drug and anti-drug antibody complexes; (g) contacting the solution of step (f) with the first drug coated substrate of step (b) and incubating the solution with the drug coated substrate for a time period to form an immobilized complex between the drug coated on the substrate and the anti-drug antibody present in the solution; (h) washing the formed complex on the first substrate with a wash buffer to remove disassociated drug originally present in the sample from the solution; (i) treating the complex of step (h) with an acid or a base to disassociate the complex to form a second solution of the anti-drug antibody, wherein the acid treatment disrupts binding of the anti-drug antibody to the immobilized drug and wherein the drug remains immobilized upon the first substrate; and contacting the solution with a second substrate; (j) detecting the presence of anti-drug antibodies, if present, by incubating the second substrate with a detection reagent. In one aspect, the method has a sensitivity for detecting levels of anti-drug antibodies before the presence of anti-drug antibodies affects one or more parameters comprising, pharmacokinetic, pharmacodynamic, safety, or efficacy. In another aspect, the method has a sensivity in terms of mass of anti-drug antibody detected per mL of sample, wherein the sensitivity comprises a range of between 10 ng/mL to 1,000 ng/mL, 100 ng/mL to 1000 ng/mL, 200 ng/mL to 1000 ng/mL, or 250 ng/mL to 500 ng/mL. In another aspect, the method has a sensitivity of at least 250 ng/mL. In another aspect, the method has a sensitivity of at least 100 ng/mL.

Another embodiment described herein is a method for reducing drug target interference in an immunogenicity assay comprising obtaining a sample having a drug and a drug target and heating the sample, wherein the heating step reduces binding of the drug to the target. In one aspect, the immunogenicity assay is an anti-drug antibody assay. In another aspect, the immunogenicity assay is a neutralizing antibody assay. In another aspect, the amount of drug target interference is reduced by at least about 10% to at least about 90%. In another aspect, the amount of drug target interference is reduced by at least about 50%. In another aspect, the immunogenicity assay is an anti-drug antibody assay. In another aspect, the immunogenicity assay is a neutralizing antibody assay. In another aspect, the immunogenicity assay is a pharmacokinetic assay. In another aspect, the immunogenicity assay is a biomarker assay. In another aspect, the immunogenicity assay is a toxicological assay. Another embodiment described herein is a method for detecting anti-drug antibodies that are antigenic to a drug in a sample comprising: (a) obtaining a sample suspected to have one or more anti-drug antibodies; (b) coating a first substrate with the drug to create an immobilized drug coated substrate; (c) heating the sample for a time period, wherein the heating of the sample reduces drug target binding to the drug; (d) cooling the sample of step (c); (e) treating the sample of step (d) with an acid or a base to disassociate any drug and anti-drug antibodies to form a solution of disassociated drug and anti-drug antibody complexes; (f) contacting the sample of step (e) with the first drug coated substrate of step (b) to form an immobilized complex between the drug coated on the substrate and the anti-drug antibody present in the sample; and (g) detecting the presence of anti-drug antibodies, if present, with a detection reagent.

### BRIEF DESCRIPTION OF THE DRAWINGS

FIG. 1 is a bar graph quantifying the amount of detected drug target from various human serum samples bound to a captured drug in a plate assay following different sample treatment conditions including boiling at 5 seconds or 15 seconds, centrifugation, and EDTA treatment.

FIG. 2 is a bar graph quantifying the amount of detected ADA and drug target following different length boiling times at different concentrations of ADA.

FIG. 3 is a bar graph quantifying the amount of detected drug target from various human serum samples bound to captured drug in a plate assay following different sample treatment conditions including boiling at 15 seconds, centrifugation, and EDTA treatment.

### DETAILED DESCRIPTION

The term “anti-drug antibodies” or “ADAs” as used herein refers to antibodies that bind specifically to any region of a drug. For example, an ADA may be an antibody or fragment thereof, which may be directed against any region of a drug antibody, e.g., the variable domain, the constant domains, or the glycostructure of the antibody. Such ADAs may occur during drug therapy as an immunogenic reaction of a patient. An ADA may be one of any human immunoglobulin isotype (e.g., IgM, IgE, IgA, IgG, IgD) or IgG subclass (IgG1, 2, 3, and 4). ADAs include ADAs from any animal source, including, for example, human or non-human animal (e.g., veterinary) sources. The term “neutralizing antibody” or “NAB” refers to an antibody that binds to an endogenously produced molecule, e.g., an antibody, nucleic acid, peptide, polypeptide, peptidomimetic, carbohydrate, or lipid. For example, a NAB may be an endogenously produced protein, such as, for example, erythropoietin, or insulin. The NAB may or may not reduce (e.g., neutralizes) at least one biological activity of the endogenously produced molecule.

The term “patient” refers to any subject including mammals and humans. The patient may have a disease or be suspected of having a disease and as such is being treated with a drug. The term “subject,” as used herein, refers to any animal (e.g., a human or non-human animal subject). In some instances, the subject is a mammal. In some instances, the term “subject,” as used herein, refers to a human (e.g., a man, a woman, or a child). In some instances, the term “subject,” as used herein, refers to a laboratory animal of an animal model study. The patient or subject may be of any age, sex, or combination thereof.

The terms “biological sample” or “sample” as used herein refers to a sample obtained or derived from a patient that comprises patient immunoglobulin and may therefore be referred to as an immunoglobulin sample. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), urine, fluids of the eye (e.g., vitreous fluid, aqueous humor), lymph fluid, lymph node tissue, spleen tissue, bone marrow, and an immunoglobulin enriched fraction derived from one or more of these tissues. In some embodiments the sample is, or comprises blood serum or is an immunoglobulin enriched fraction derived from blood serum or blood. The sample is, or can be derived (obtained) from, a bodily fluid or body tissue. In some embodiments, the sample is obtained from a subject who has been exposed to the drug, such as repeatedly exposed to the same drug. In other embodiments, the sample is obtained from a subject who has not recently been exposed to the drug, or obtained from the subject prior to the planned administration of the drug.

The term “substrate” as used herein refers to any material or macromolecular complex to which an ADA or drug material (e.g., an antibody, nucleic acid, peptide, polypeptide, peptidomimetic, carbohydrate, lipid, or an organic or inorganic small molecule compound) may bind. The composition and/or surface of the substrate should allow for binding of an ADA or drug material complexed or uncomplexed. The composition and/or surface of the substrate should further allow for binding under acidic conditions (or basic conditions) that allow for dissociation of the ADA/drug complexes. In some embodiments, these substrates have a high loading capacity, which improves sensitivity, thus allowing for detection of ADAs and/or drug materials present in relatively low concentrations. Examples of commonly used substrates include, but are not limited to, carbon surfaces (e.g., a porous or high bind carbon plate), glass surfaces, silica surfaces, plastic surfaces, metal surfaces, surfaces containing a metallic or chemical coating, membranes (e.g., nylon, polysulfone, silica), micro-beads (e.g., latex, polystyrene, or other polymer), porous polymer matrices (e.g., polyacrylamide gel, polysaccharide, polymethacrylate), and substrates comprising cellulosic fibers (e.g., cellulose sponges, cellulose paper). The substrate may be a biosensor chip, microarray, or lab-on-chip capable of sensing a target molecule. Any kind of biosensor that is capable of sensing specific binding to the biosensor chip is applicable, including commercially available biosensors, such as the biosensors produced by Biacore.

As used herein, an entity (e.g., antibody, anti-drug antibody, drug, protein, enzyme, antibody, antibody fragment, multiple domain biotherapeutics (e.g., antibody drug conjugates), or related species) that is modified by the term “labeled” includes any entity that is conjugated with another molecule or chemical entity a that is empirically detectable (e.g., “detectable label”). Chemical species suitable as labels for labeled-entities include, but are not limited to, enzymes, fluorescent dyes; quantum dots; optical dyes; luminescent dyes; and radionuclides.

The term “drug tolerance” as used herein is defined as the maximal amount of free drug in a sample that still results in a detectable ADA signal.

The term “target interference” or “drug target interference” as used herein is defined as the target of a therapeutic or a drug, which interferes with the accurate detection of an ADA. The target interference can lead to increased false positive or false negative results dependent upon the immunogenicity assay.

The term “minimal required dilution” or “MRD” refers to a sample dilution that yields a signal that is close to the assay diluent. Selection of the minimal required dilution allows for the highest signal-to-noise ratio.

The term “cut point,” as used herein refers to the level of response in the selected assay, which has been determined to define the sample as positive or negative for an ADA. A suitable cut point identifies samples producing a signal that is beyond the variability of the assay (see e.g., FDA April 2016, Assay Development and Validation for Immunogenicity Testing of Therapeutic Protein Products, Draft Guidance for Industry for further guidance on establishing and selecting cut points in immunogenicity assays).

The term “sensitivity” as used herein refers to the lowest concentration at which an antibody preparation consistently produces a positive result or one that is equal to the cutpoint of the assay. The sensitivity is typically expressed as the mass of antibody per mL of sample. In some aspects, the assays described herein enable detection of ADAs prior to an ADA in a patient producing any altered pharmacokinetic, pharmacodynamic, safety, or efficacy profiles.

The term “specificity” as used herein refers to the ability of the methods disclosed herein to detect ADAs, which bind to therapeutic proteins and not any of the assay components.

The term “selectivity” as used herein refers to the ability of the methods disclosed herein to correctly identify drug specific ADAs out of a complex biological sample.

The term “drug” or “therapeutic” as used herein refers to any natural or unnatural compound that elicits a biological effect including medicinal, performance-enhancing, and/or intoxicating effects when introduced into the body of a human or other animal. Thus, the drug may be a small molecule compound, a biologic such as a protein, nucleic acid (e.g., DNA or RNA). For example, the drug can be an organic or inorganic small molecule compound or a biologic therapeutic (e.g., an antibody (e.g., a drug antibody) or fragment thereof, multiple domain biotherapeutics, nucleic acid, peptide, polypeptide, peptidomimetic, carbohydrate, or lipid), as long as the drug is immunogenic and capable of eliciting an immune response. The term “drug antibody” denotes an antibody that can be administered to an individual for the treatment of a disease and as used herein distinguishes such antibodies from ADAs.

The approaches disclosed herein have been shown to eliminate drug target interference and improve drug tolerance in ADA assays. In practice, this methodology can be applied to reduce or eliminate interferences in any type of immunoassay. This methodology can used for any ligand binding assays, for example, ADA, PK, and biomarker assays. The methods described herein can be applied to ligand binding assays to test for neutralizing antibodies (NABs). The ligand binding assays can include competitive inhibition of drug binding to drug target.

The methods described herein comprise heating of the sample being assayed. Heating the sample reduces or eliminates drug target binding to the drug. Without being bound by any theory, it is thought that the heating of the sample denatures the target protein to an extent to reduce its binding to the drug, while not affecting the binding of an ADA to the drug. This finding is unexpected because it generally would be expected that heating a biological sample to an extent that would denature a drug/target interaction would also decrease the binding of an ADA to its target. Unexpectedly, the inventors discovered that ADAs could be detected with a higher sensitivity and specificity when the sample was heated prior to being assayed in comparison to sample preparation wherein the sample was not heated prior to being assayed.

Therefore, one embodiment described herein is the application of a heating step during an anti-drug antibody assay that reduces drug target interference and improves the quality of the assay. Another embodiment described herein is the application of a heating step to reduce drug target interference in conjunction with additional methods for improving drug tolerance as described herein. In some embodiments, the additional methods include affinity capture elution (ACE), biotin-drug extraction with acid dissociation assay (BEAD), solid-phase extraction with acid dissociation (SPEAD), or precipitation and acid dissociation (PanDA), or a combination of those methods in part or in full.

Another embodiment described herein is a method for detecting anti-drug antibodies that are antigenic to a drug in a sample. In one aspect, the method includes obtaining a biological sample as defined herein, which is suspected to have one or more ADAs. In another aspect, the method includes heating the sample for a time period, wherein the heating of the sample reduces drug target binding to the drug. In some aspects, the method may be carried out in conjunction with any of the steps of a conventional ADA detection assay, including a bridging ADA assay, an ACE assay, a BEAD affinity capture elution assay, a SPEAD assay or PanDA assay as known in the art and those described herein.

Another embodiment described herein is a method for detecting an ADA that is antigenic to a drug in a sample comprising: obtaining a sample suspected to have one or more anti-drug antibodies; coating a first substrate with the drug to create an immobilized drug coated substrate; heating the sample for a time period, wherein the heating of the sample reduces drug target binding to the drug; and contacting the sample suspected of having an ADA with the first drug coated substrate to form an immobilized complex between the drug coated on the substrate and the anti-drug antibody present in the sample; and detecting the presence of ADA, if present, with a detection reagent.

Another embodiment described herein is a method for detecting ADAs that are antigenic to a drug in a sample that includes obtaining a sample suspected to have one or more anti-drug antibodies and coating a first substrate with the drug to create an immobilized drug coated substrate. In one aspect, the method further includes heating the sample to a temperature described herein, wherein the heating step reduces drug target binding to the drug, and cooling the heated sample. In another aspect, the method further includes diluting the sample to the minimum required dilution (e.g., in an antibody blocking buffer). In another aspect, the method further includes treating the diluted sample with a disassociation reagent (e.g., an acid or a base) to disassociate any drug and ADAs. In another aspect, the disassociated drug and ADAs are present as a solution and the solution is put in contact with the first drug-coated substrate and incubated with the drug-coated substrate for a time period to form an immobilized complex between the drug coated on the substrate and the ADA present in the solution. In another aspect, the formed complex on the first substrate between the ADA and immobilized drug is washed with a suitable wash buffer to remove disassociated drug originally present in the sample from the solution. In another aspect, the method further includes treating the complex on the first substrate with a disassociation reagent (e.g., an acid or a base) to disassociate the complex to form a second solution of the anti-drug antibody, wherein the acid treatment disrupts binding of the anti-drug antibody to the immobilized drug and wherein the drug remains immobilized upon the first substrate. In another aspect, the method further includes contacting the solution with a second substrate (e.g., a high-bind carbon plate), which binds the ADA; and detecting the presence of anti-drug antibodies, if present, by incubating the second substrate with a detection reagent.

Another embodiment described herein is a method for determining whether a sample is positive or negative for having immunogenic ADAs. In one aspect, the method includes detecting an ADA as described herein and characterizing the sample as positive for having an ADA if the detected signal is above a pre-determined cut point or negative and not having an ADA if the detected signal is below a pre-determined cut point.

Another embodiment described herein is a method for reducing drug target interference in an immunogenicity assay. In one aspect, the method includes obtaining a sample having a drug and a drug target and heating the sample, wherein the heating step reduces binding of the drug to the target as further described herein. The assay may be any assay in which the presence of an antibody is being assayed and where drug/target interference is suspected to be present (e.g., an ADA assay or a NAB assay). The amount of drug target interference in a sample is reduced by application of the heating step by a certain percentage compared to a control not having any heating step. Thus, in some aspects, the amount of drug target interference is reduced by about 10% to about 99%, including each integer within the specified range. In one aspect, the amount of drug target interference is reduced by about 30% to about 99%, including each integer within the specified range. In another aspect, the amount of drug target interference is reduced by at least about 5%. In another aspect, the amount of drug target interference is reduced by at least about 10%. In another aspect, the amount of drug target interference is reduced by at least about 50%. In another aspect, the amount of drug target interference is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%.

Another embodiment described herein comprises performing a titration assay to determine the quantity of ADA present in a sample. The titration assay is generally the same assay that was used to perform the detection of the ADA in a sample. An exemplary method for titering the ADA includes sequentially diluting a sample and conducting the assay used to initially detect an ADA. The sample is diluted sequentially until the point at which the detection signal falls below the cut point. Alternatively, the titering method may include extrapolating a dilution curve to a pre-established assay cut point.

Another embodiment described herein includes performing a confirmation test to confirm the presence of an ADA, and reduce the likelihood of any false positive result. Methods for confirming the presence of an ADA generally include performing the ADA detection methods described herein with competitive inhibition of a labelled ADA detector, which is typically the labeled drug to which the ADA is antigenic. In this confirmation assay, an unlabeled detector (e.g., the drug) is added in conjunction with labeled detector (e.g., the drug) and the amount of signal inhibition in samples having the unlabeled detector is quantified and compared to those having not having the unlabeled detector. If the signal is inhibited to an established confirmatory cut point, then they will be confirmed as positive.

Some embodiments described herein are methods for detecting anti-drug antibodies that are antigenic to a drug in a sample. The methods include heating the sample for a time period during which the heating of the sample reduces drug target binding to the drug. As described below, the time period at which the sample is heated indicates the amount of time the sample is at the maximal temperature. In some aspects, the time period that the sample is heated is from about 1 second to about 72 hours, including every iteration of time within the specified range. In one aspect, the time period that the sample is heated is from about 1 second to about 24 hours, including every iteration of time within the specified range. In another aspect, the time period that the sample is heated is from about 1 second to about 2 hours, including every iteration of time within the specified range. In another aspect, the time period that the sample is heated is from about 1 second to about 30 minutes, including every iteration of time within the specified range. In another aspect, the time period that the sample is heated is from about 1 second to about 5 minutes, including every iteration of time within the specified range. In another aspect, the time period that the sample is heated is about 1 second, about 2 seconds, about 3 seconds, about 4 seconds, about 5 seconds, about 10 seconds, about 15 seconds, about 20 seconds, about 25 seconds, about 30 seconds, about 40 seconds, about 50 seconds, about 60 seconds, about 70 seconds, about 80 seconds, about 90 seconds, about 100 seconds, about 110 seconds, or about 120 seconds.

In some embodiments, the sample is heated for a time period sufficient to reach a specific temperature. In one embodiment, the sample is heated for a time period sufficient to reach the maximal temperature that the sample is to be heated. For example, it may take 10 seconds to heat a sample to the maximal temperature. The time sufficient to achieve the maximal heating temperature is dependent upon sample volume, baseline temperature, heating equipment, plastic ware holding the sample(s) and other factors. Exemplary time periods sufficient to reach a specific temperature may include time periods of about 1 second to about 2 hours, including every iteration of time within the specified range. In another aspect, the time period sufficient to reach a specific temperature may include a time period of about 1 second to about 5 minutes, including every iteration of time within the specified range. In another aspect, the time period sufficient to reach a specific temperature may be about 1 second, about 2 seconds, about 3 seconds, about 4 seconds, about 5 seconds, about 10 seconds, about 15 seconds, about 20 seconds, about 25 seconds, about 30 seconds, about 40 seconds, about 50 seconds, about 60 seconds, about 70 seconds, about 80 seconds, about 90 seconds, about 100 seconds, about 110 seconds, or about 120 seconds.

In some embodiments, the sample is heated to a specific temperature. For example, the temperature may be from about 30° C. to about 100° C., including each integer within the specified range. In some aspects, the temperature is from about 40° C. to about 100° C., including each integer within the specified range. In some aspects, the temperature is from about 50° C. to about 95° C., including each integer within the specified range. In some aspects, the temperature is from about 60° C. to about 95° C., including each integer within the specified range. In some aspects, the temperature is from about 60° C. to about 85° C., including each integer within the specified range.

In some aspects, the temperature is from about 60° C. to about 85° C., including each integer within the specified range. In some aspects the temperature is about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C.

In some embodiments, the sample is cooled after having been heated. For example, after heating, the sample can be cooled and stored at an appropriate temperature. In one aspect, the sample can be cooled and stored at about room temperature, about 4° C., about 0° C., −20° C., or −80° C. for a period of time (such as overnight or until needed for a subsequent step). In one aspect, the sample is cooled after heating and then stored at an appropriate temperature for a period of time until used in a subsequent assay step.

In some embodiments, the sample is cooled over a time period after being heated. For example, it may take 10 seconds to cool a sample to the desired temperature, which would be the cooling time. The time to achieve the desired cooled temperature after heating is dependent upon sample volume, the sample temperature, cooling temperature, equipment used to cool the sample, and other factors. Thus, in some aspects, the sample is cooled over a time period ranging from about 1 second to about 2 hours, including every iteration of time within the specified range. In another aspect, the sample is cooled over a time period ranging from 1 second to about 30 minutes, including every iteration of time within the specified range. In another aspect, the sample is cooled over a time period ranging from about 1 second to about 5 minutes, including every iteration of time within the specified range. In another aspect, the sample is cooled over a period time of about 1 second, about 2 seconds, about 3 seconds, about 4 seconds, about 5 seconds, about 10 seconds, about 15 seconds, about 20 seconds, about 25 seconds, about 30 seconds, about 40 seconds, about 50 seconds, about 60 seconds, about 70 seconds, about 80 seconds, about 90 seconds, about 100 seconds, about 110 seconds, or about 120 seconds.

In some embodiments, the sample is cooled for a time period sufficient to reach a specific temperature. In one embodiment, the sample is cooled for a time period sufficient to reach the minimal temperature that the sample is to be cooled. For example, it may take 10 seconds to cool a sample to the minimal temperature. The time sufficient to achieve the minimal cooling temperature is dependent upon sample volume, baseline temperature, heating equipment, plastic ware holding the sample(s) and other factors. Thus, in some aspects, the sample is cooled over a time period sufficient to reach a specific temperature of about 1 second to about 2 hours, including every iteration of time within the specified range. In another aspect, the sample is cooled over a time period sufficient to reach a specific temperature of about 1 second to about 5 minutes, including every iteration of time within the specified range. In another aspect, the sample is cooled over a time period of about 1 second, about 2 seconds, about 3 seconds, about 4 seconds, about 5 seconds, about 10 seconds, about 15 seconds, about 20 seconds, about 25 seconds, about 30 seconds, about 40 seconds, about 50 seconds, about 60 seconds, about 70 seconds, about 80 seconds, about 90 seconds, about 100 seconds, about 110 seconds, or about 120 seconds in order to reach a specific temperature.

In some embodiments, the sample is cooled to a specific temperature. In some aspects, the temperature is from about −80° C. to about 35° C., including each integer within the specified range. In some aspects, the temperature is from about −80° C. to about 20° C., including each integer within the specified range. In some aspects, the temperature is from about −20° C. to about 4° C., including each integer within the specified range. In some aspects, the temperature is from about 0° C. to about 10° C., including each integer within the specified range. In some aspects, the temperature is from about 0° C. to about 4° C., including each integer within the specified range. In some aspects, the temperature is about −80° C., about −20° C., about 0° C., about 4° C., about 10° C., about 15° C., about 20° C., about 25° C., about 30° C., or about 35° C. In some aspects, the sample is cooled to a specific temperature and then stored at about 0° C., about 4° C., about −20° C., or about −80° C. for a time period.

Methods for heating and cooling samples are known to the skilled person in the art. For example, the sample may be heated by placing it in a warm to hot water bath for a time period to reach the desired temperature. Conversely, samples may be cooled by placing the sample into a cool to cold water or ice bath for a time period to reach the desired temperature. Automated methods include the use of a thermocycler, in which multiple samples may be cooled and heated at specific rates and temperatures in a highly controlled environment. In some embodiments, it is contemplated that the sample is heated at the maximal rate at which the thermocycler is capable of heating. In other embodiments, the sample is cooled at the maximal rate at which the thermocycler is capable of cooling.

In some embodiments, the sample is diluted to the minimum required dilution (MRD), which prevents matrix components and components of the sample from contributing to non-specific background signals. The MRD is typically determined from ADA negative samples of untreated patients. In some aspects, the MRD is from about 1:2 to about 1:500, including all iterations of ratios within the specified range. In some aspects, the MRD is from about 1:5 to about 1:100, including all iterations of ratios within the specified range. In some aspects, the MRD is from about 1:10 to about 1:50, including all iterations of ratios within the specified range. In one aspect, the MRD is about 1:10, about 1:20, about 1:30, about 1:40, about 1:50, about 1:60, about 1:70, about 1:80, about 1:90, or about 1:100.

In some embodiments, the sample is further diluted to the MRD in a blocking buffer or other solution or solvent. In one aspect, the sample is further diluted to the MRD in a blocking buffer. The use of a blocking buffer can help prevent non-specific interactions between antibodies. Suitable blocking agents include bovine serum albumin, mammalian sera (e.g., human, bovine, calf, horse, goat, rabbit, or mouse), casein, dried milk, commercial blocking agents (e.g., Blocker A from Meso Scale Discovery®), or a combination thereof. The blocking agent can be used at a concentration ranging from 1% to about 20% (w/v) in a suitable buffer. In some aspects, the blocking agent is used at a concentration of 5% (w/v). Suitable buffers include, but are not limited to, tris-buffered saline and polysorbate (TBST) and phospho buffered saline (PBS) and the like.

In some embodiments, a chelator is added to the sample prior to heating the sample. In some aspects, the sample is diluted with a buffer comprising the chelator. Any chelators that do not affect the ability to detect an ADA or NAB are suitable for use. Exemplary and non-limiting chelators include, but are not limited to, ethylenediaminetetraacetic acid (EDTA), ethylene glycol tetraacetic acid (EGTA), 2,2′-bipyridyl, dimercaptopropanol, salicylic acid, triethanolamine, nitrilotriacetic acid, ortho-phenanthroline, or a combination thereof. In another aspect, the chelator is present at a concentration of about 0.1 mg/mL to about 10 mg/mL.

The dilution agent may comprise a physiologically compatible salt such as an alkali metal salt (e.g., sodium chloride, potassium chloride, magnesium chloride, etc.) and an organic acid. The organic acid may be a weak acid having a pKₐ of about 2-5. Exemplary acids include, but are not limited to, those such as formic acid, acetic acid propionic acid, butyric acid, valeric acid, caproic acid, oxalic acid, lactic acid, malic acid, citric acid, benzoic acid, phosphoric acid, or carbonic acid.

In some embodiments, a chelator is added to a sample prior to diluting the sample to the MRD. The sample may then be diluted further to the MRD in a suitable buffer (e.g., an antibody blocking buffer). In some aspects, the chelator is added to achieve a concentration of about 0.1 mg/mL to about 10 mg/mL, including each integer within the specified range. In some aspects, the chelator is added to achieve a concentration of about 0.1 mg/mL to about 5 mg/mL, including each integer within the specified range. In some aspects, the chelator is added to achieve a concentration of about 0.1 mg/mL to about 2 mg/mL, including each integer within the specified range. In some aspects, the chelator is added to achieve a concentration of about 0.1 mg/mL to about 1 mg/mL, including each integer within the specified range. In some aspects, the chelator is added to achieve a concentration of about 0.1 mg/mL, about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 6 mg/mL, about 7 mg/mL, about 8 mg/mL, about 9 mg/mL, or about 10 mg/mL.

In some embodiments, the sample or a substrate having an ADA/drug complex is treated with a disassociation reagent to disrupt ADA/drug complexes. Thus, the disassociation reagent may be any chemical or compound, which disrupts the complex, but does not denature or prevent an ADA from binding to the drug in further assay steps. In some embodiments, the ADA/drug complex is disassociated with an acid or a base to disrupt ADA/drug complexes.

Suitable acids for use in the methods disclosed herein comprise organic acids or amino acids. Alternatively or in addition, the acid comprises an inorganic acid. The acid used in the dissociation step may comprise a mixture of an organic acid and an inorganic acid. Non-limiting examples of organic acids include, for example, citric acid, isocitric acid, glutamic acid, acetic acid, lactic acid, formic acid, oxalic acid, uric acid, trifluoroacetic acid, benzene sulfonic acid, aminomethanesulfonic acid, camphor-10-sulfonic acid, chloroacetic acid, bromoacetic acid, iodoacetic acid, propanoic acid, butanoic acid, glyceric acid, succinic acid, malic acid, aspartic acid, glycine, and combinations thereof. Non-limiting examples of inorganic acids include, for example, hydrochloric acid, nitric acid, phosphoric acid, sulfuric acid, boric acid, hydrofluoric acid, hydrobromic acid, and mixtures thereof.

The amount of an acid may correspond to a concentration of between about 0.01 M to about 10 M, between about 0.1 M to about 5 M, about 0.1 M to about 2 M, between about 0.2 M to about 1 M, or between about 0.25 M to about 0.75 M of an acid or a mixture of acids. In some instances the amount of an acid corresponds to a concentration of greater than or equal to about 0.01 M, 0.05 M, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6 M, 7 M, 8 M, 9 M, or 10 M of an acid or a mixture of acids. The pH of the acid can be, for example, about 0.1, 0.5, 1.0, 1.5, 2.0, 2.5, 3.0, 3.5, 4.0, 4.5, 5.0, 5.5, 6.0, or 6.5.

Alternatively, in some embodiments, the methods described herein may comprise a base dissociation step, wherein the drug/ADA complex is disassociated. The base can comprise an organic base. Alternatively or in addition, the acid comprises an inorganic base. The base used in the dissociation step may comprise a mixture of an organic base and an inorganic base. Non-limiting examples of bases include, for example, urea, sodium hydroxide, rubidium hydroxide, cesium hydroxide, calcium hydroxide, potassium hydroxide, strontium hydroxide, barium hydroxide, zinc hydroxide, lithium hydroxide, acetone, methylamine, and ammonia, ammonia hydroxide, tris(hydroxymethyl)aminomethane (Tris base), trimethylamine, or bicarbonate salts or mixtures thereof.

Where a basic solution is used to disrupt the ADA/drug interaction, the amount of base may correspond to a concentration of between about 0.01 M to about 5 M, between about 0.1 M to about 5 M, about 0.1 M to about 1 M, between about 0.2 M to about 1 M, or between about 0.25 M to about 0.75 M of a base or a mixture of bases. In some instances the amount of a base corresponds to a concentration of greater than or equal to about 0.01 M, 0.05 M, 0.1 M, 0.2 M, 0.3 M, 0.4 M, 0.5 M, 0.6 M, 0.7 M, 0.8 M, 0.9 M, 1 M, 2M, 3M, 4M, 5M, 6M, 7M, 8M, 9M, or 10 M of a base or a mixture of bases. The pH of the base can be, for example, about 8.0, 8.5, 9.0, 9.5, 10.0, 10.5, 11.0, 11.5, 12.0, 12.5, or 13.0.

In some embodiments, the sample is contacted with an acid or base for an amount of time sufficient to dissociate preformed drug/ADA complexes. In certain instances, the sample or a substrate having a drug/ADA complex is contacted (e.g., incubated) with an acid or base for a time period ranging from about 0.1 hours to about 24 hours, e.g., about 0.2 hours to about 16 hours, about 0.5 hours to about 10 hours, about 0.5 hours to about 5 hours, or about 0.5 hours to about 2 hours. In other instances, the sample is contacted (e.g., incubated) with an acid or base for a time period that is greater than or equal to about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 hours. The sample can be contacted with an acid or a base at any temperature that is generally compatible with the method, e.g., 4° C., room temperature (RT), or 37° C. Room temperature can be, for example, 22° C. to 26° C., e.g., 23° C., 24° C., or 25° C.

In some embodiments, a first and a second substrate is used to capture an ADA. In some aspects, the first substrate is coated with a drug to which the ADA is antigenic. In some aspects, the second substrate is configured for binding an ADA. Exemplary and non-limiting substrates include a carbon surface, glass surface, silica surface, metal surface, a polymeric material, a surface containing a metallic or chemical coating, a membrane, a bead (e.g., a micro-bead), a porous polymer matrix, a substrate comprising cellulosic fibers, or any combinations thereof. The substrate can comprise a polymeric material, wherein the polymeric material is selected from the group consisting of polystyrene, polyvinyl chloride, polypropylene, polyethylene, polyamide, and polycarbonate. The substrate can be configured to bind molecules which is hydrophobic, hydrophilic or mixed hydrophobic and hydrophilic e.g., PolySorp™, MediSorp™, MaxiSorp™, and MultiSorp™ plates available from (Nunc and Thermo Fisher Scientific). The substrate can be in the form of a plate, a bead, a tube (e.g., a 0.2-1 mL tube), a multi-well plate (e.g., anywhere from 6-384 wells). In some aspects, the first substrate is a MaxiSorp™ plate. In some aspects, the second substrate has a hydrophobic coating surface (e.g., a Meso Scale Discovery® standard SECTOR® plate.

In some embodiments, the presence of an ADA is detected with an entity that is labeled. In some aspects, antibodies, anti-drug antibodies, and drug are conjugated to a detectable label. The detectable label is any reagent, which can be detected. For example, a label can be a hapten, radioactive isotope, an enzyme, a fluorescent label, a chemiluminescent label, and electrochemiluminescent label, a first member of a binding pair, and a substrate for an enzymatic detection reaction. In one aspect, the label is an electrochemiluminescent label (e.g., ruthenium).

The detectable label may comprise a fluorophore, wherein the fluorophore comprises one or more of green fluorescent protein, blue fluorescent protein, red fluorescent protein, fluorescein, fluorescein 5-isothiocyanate (FITC), cyanine dyes (Cy3, Cy3.5, Cy5, Cy5.5, Cy7), Bodipy dyes (Invitrogen) and/or Alexa Fluor dyes (Invitrogen), dansyl, Dansyl Chloride (DNS-C1), 5-(iodoacetamide)fluorescein (5-IAF, 6-acryloyl-2-dimethylaminonaphthalene (acrylodan), 7-nitrobenzo-2-oxa-1,3, -diazol-4-yl chloride (NBD-C1), ethidium bromide, Lucifer Yellow, rhodamine dyes (5-carboxyrhodamine 6G hydrochloride, Lissamine rhodamine B sulfonyl chloride, rhodamine-B-isothiocyanate (RITC (rhodamine-B-isothiocyanate), rhodamine 800); tetramethylrhodamine 5-(and 6-)isothiocyanate (TRITC)), Texas Red™, sulfonyl chloride, naphthalamine sulfonic acids including but not limited to 1-anilinonaphthalene-8-sulfonic acid (ANS) and 6-(p-toluidinyl)naphthalen-e-2-sulfonic acid (TNS), Anthroyl fatty acid, DPH, Parinaric acid, TMA-DPH, Fluorenyl fatty acid, Fluorescein-phosphatidylethanolamine, Texas red-phosphatidylethanolamine, Pyrenyl-phoshatidylcholine, Fluorenyl-phosphatidylcholine, Merocyanine 540, Naphtyl Styryl, 3,3′dipropylthiadicarbocyanine (diS-C3-(5)), 4-(p-dipentyl aminostyryl)-1-methylpyridinium (di-5-ASP), Cy-3 lodo Acetamide, Cy-5-N-Hydroxysuccinimide, Cy-7-Isothiocyanate, IR-125, Thiazole Orange, Azure B, Nile Blue, Al Phthalocyanine, Oxaxine 1,4′, 6-diamidino-2-phenylindole. (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARF-6, BAPTA, coumarin, phytofiuors, or Coronene.

The detectable label may comprise an enzyme that catalyzes a reaction producing a detectable signal, such as production of a chromophore, including, an enzyme selected from the group consisting of alkaline phosphatase, beta-galactosidase, horse radish peroxidase, urease and beta-lactamase, or glucose oxidase. In some embodiments, the detectable label comprises a first member of a binding pair or a second member of a binding pair, wherein the binding pair is selected from the group consisting of biotin/streptavidin, biotin/avidin, biotin/neutravidin, biotin/captavidin, epitope/antibody, protein A/immunoglobulin, protein G/immunoglobulin, protein L/immunoglobulin, GST/glutathione, His-tag/Nickel, antigen/antibody, FLAG/M1 antibody, maltose binding protein/maltose, calmodulin binding protein/calmodulin, enzyme-enzyme substrate, and receptor-ligand binding pairs.

The detectable label may comprise a first member of a binding pair; and the second member of the binding pair may be conjugated to an enzyme, an antibody epitope, an antigen, a fluorophore, a chromophore, a radioisotope, a nanoparticle, a member of a second binding pair, and a metal chelate. In other embodiments, the detectable label comprises a first member of a binding pair, wherein the first member of the binding pair is biotin and the second member of the binding pair is selected from the group consisting of streptavidin, avidin, neutravidin, or capravidin, and the second member of the binding pair conjugated to an enzyme.

In some embodiments, the samples or immobilized ADA, immobilized drug, or immobilized ADA/drug complex is washed between steps. Any suitable wash buffer used in immunological assays may be used such as phosphate buffered saline (PBS), Tris-buffered saline (TB S) and those containing polysorbate 20 (e.g., Tween® 20).

In some embodiments, the methods disclosed herein are used to determine whether ADAs have been formed against a drug antibody. Non-limiting examples of drug antibodies include, for example, an antibody selected from muromomab-CD3, abciximab, rituximab, daclizumab, basiliximab, palivizumab, infliximab, trastuzumab, etanercept, gemtuzumab, fresolimumab, alemtuzumab, ibritomomab, adalimumab, alefacept, omalizumab, tofacitinib, tositumomab, efalizumab, cetuximab, bevacizumab, natalizumab, ranibizumab, panitumumab, eculizumab mepolizumab, necitumumab, blinatumomab, nivolumab, dinutuximab, secukinumab, evolocumab, pembrolizumab, ramucirumab, vedoluzumab, siltuximab, opinutuzumab, adotrastuzumab emtansine, raxibacumab, pertuzumab, brentuximab, belimumab, ipilimumab, denosumab, tocilizumab, ofatumumab, canakinumab, golimumab, ustekinumab, catumaxomab, or certolizumab.

In some embodiments, the methods provided herein can be performed on either a manual or automated instrument platform, depending on the number of samples to be tested.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any and all variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

### Heating to Reduce Drug Target Interference in Immunogenicity Assays

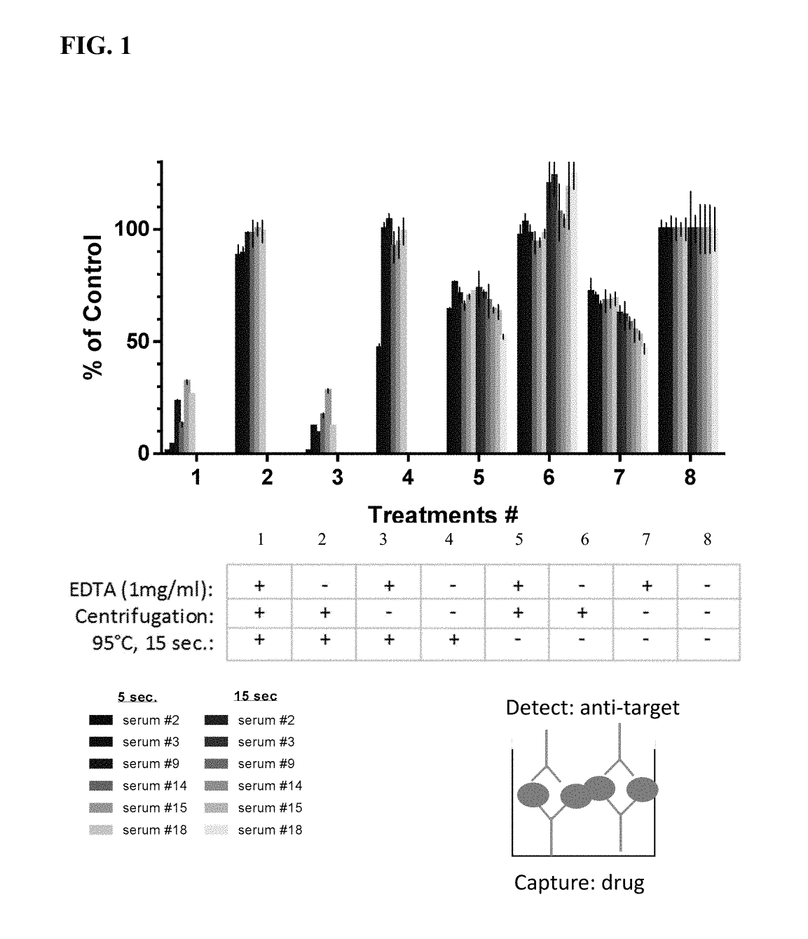

Testing was undertaken to determine if heating of samples could reduce drug target interference. In these experiments, drug was coated and immobilized upon a substrate and used as a capture reagent to capture a target. The target was subsequently detected with an antibody specific to the target (see scheme in FIG. 1, lower right panel). Accordingly, if the target was inactivated then less of it would be detected upon the drug bound substrate and a lower signal would be generated by the detection reagent. Serum samples containing a target protein were boiled for 5 seconds or 15 seconds in the presence or absence of 1 mg/mL EDTA with or without centrifugation. The samples were then contacted with the drug-coated substrate. Experiments 1-8 tested the effects of EDTA, centrifugation, and boiling target inactivation. Experiments 1-4 were performed with 5-second boiling, whereas experiments 5-8 had 15-second boiling. The data provided in FIG. 1 shows that there was a significantly reduced detection of target protein in the plate assay in all samples that were boiled; the left bars are the 5-second experiments and the right bars are the 15-second experiments. This result indicates that boiling inactivated target protein and that the target protein was unable to conjugate with the bound drug because of reduced detection of the target in the plate assay.

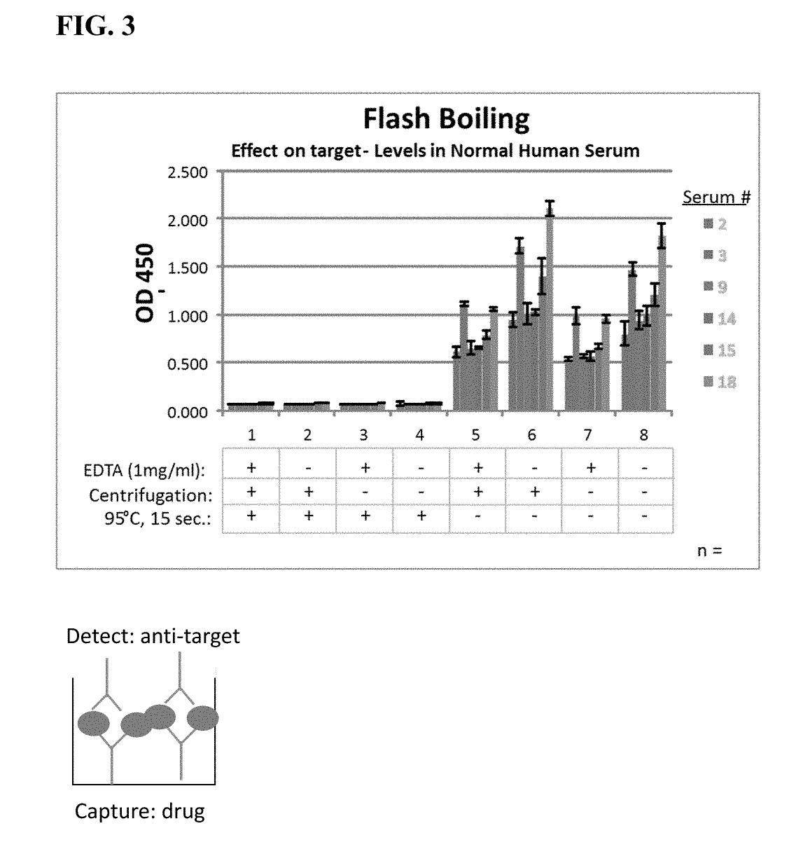

Additional testing is shown in FIG. 3 where different normal human serum samples were diluted with a diluent having 25 mM citrate pH 6.0, 150 mM NaCl, and 3.4 mM EDTA or no EDTA and boiled for target inactivation for 15 seconds. As with the experiment described above, an immobilized drug was used to capture the drug target and a labelled target specific antibody was used for detection of the target (see scheme in FIG. 3, lower left panel). Experiments 1-8 show the effects of EDTA, centrifugation, and boiling target inactivation in normal human serum. Similar to the results shown in FIG. 1, boiling significantly reduced the detection of the target protein. These results indicate that heating a sample inactivate target protein and reduce target interference in ADA immunogenicity assays.

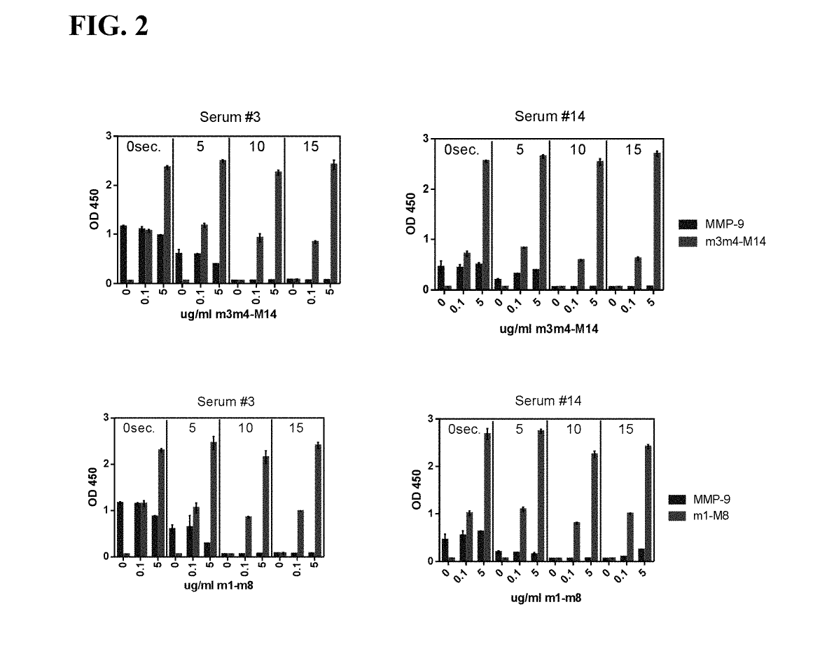

Further testing was set up to determine if heating would also inactivate any ADAs present in a sample. In this experiment, matrix metallopeptidase 9 (MMP-9) was used as a target protein and m3m4-M14 or m1-m8 at 0 μg/mL, 0.1 μg/mL, and 5 μg/mL was used as a control antibody, which served as an ADA. In this experiment, the samples were boiled for 0 seconds, 5 seconds, 10 seconds, and 15 seconds. The results of this experiment are provided in FIG. 2, which surprisingly shows that even after 15 seconds of boiling, the ADA (m3m4-M14 or m1-m8) was not inactivated whereas the target protein was completely inactivated. This result is unexpected because 15 seconds of boiling would have been predicted to inactivate at least to some extent the ADA, which was not observed.

### Example 2

### Exemplary Protocol for the Detection of ADAs Antigenic to an Antibody Therapeutic Test Drug

A method was developed to detect anti-drug antibodies (ADA) against a protein therapeutic test drug in human serum samples. The detection of ADAs is problematic because of the tendency of the test drug's target to be present at relatively high (up to 2 μg/mL) levels as both monomer and dimer, as well as other complexed species. Multiple acidification steps were further introduced to improve the drug tolerance of the assay. The first acidification reduces ADA binding to a drug and also drug binding to a target in the sample. A second acidification step is used to selectively release ADA and not additional drug from a bound ADA/drug complex provided on a substrate for detection, which is expected to improve drug tolerance. In addition, it was surprisingly discovered that the heating of samples reduced target interference in the assay without denaturing the ADAs in the sample and reduced the detected ADA signal as shown in FIG. 2.

### Equipment:

TABLE 1
Equipment used during ADA assay detection of a test drug

| Equipment | Model |
|---|---|
| Thermal Cycler | MAXYGENE II or equivalent |
| MSD ® Plate Reader | Sector Imager 6000 or equivalent |
| Bench Top Microfuge | MicroCentrifuge SD or equivalent |
| Plate Shaker | Lab Line Titer or equivalent |
| Plate Washer | BioTek Elx405 or equivalent |

### Reagents:

TABLE 2
Reagents used during ADA assay detection of a test drug

| Reagents | Vendor/Supplier | Lot Number |
|---|---|---|
| Test drug (42 mg/mL) | N/A | N/A |
| AB45m1-m8 (m1-m8, 0.34 mg/mL) | LakePharma | 6290-839392 |
| Ru-test drug (1.14 mg/mL) | N/A | N/A |
| HPC (2500 ng/mL) | | |
| LPC (100 ng/mL) | | |
| Human serum pool | Bioreclamation | |

TABLE 3
Additional reagents used during ADA assay detection of
a test drug

| Reagents | Vendor/Supplier | Lot Number |
|---|---|---|
| Maxisorp ™ plate | NUNC | 439454 |
| MSD ® standard plate | MSD ® | L15XA-3/L11XA-3 |
| Round bottom polypropylene | Corning/Costar | 3365 |
| plate | | |
| 2× Sample Diluent (50 mM | N/A | N/A |
| citrate, 300 mM NaCl, 2 mg/mL | | |
| EDTA, pH 6.0) | | |
| 1× PBS | | |
| Wash Buffer (PBS, 0.05% | | |
| Tween ® 20; PBST) | | |
| Blocking Buffer (5% Blocker | | |
| A in PBS) | | |
| 2× Read Buffer | | |
| 0.5M glycine, pH 2.0 | | |
| 1M Tris, pH 9.5 | Boston | BM-324 |
| | Bioproducts | |

Abbreviations:
CV: Coefficient of Variation
HPC: High Positive Control
LPC: Low Positive Control
Lum: Luminescence signal
MRD: Minimum Required Dilution
Norm: Normalized
NC: Negative Control
PBS: Phosphate Buffered Saline
PBST: Phosphate Buffered Saline + 0.05% Tween 20
Ru: Ruthenium (also called SulfoTag)
SPC or PC: Surrogate Positive Control
QC: Quality Control
QNS: Quantity not sufficient (to assay)

### Quality Controls and Sample Preparation

Anti-test drug quality controls were prepared by spiking m1-m8 into a human serum pool at low (100 ng/mL) and high (2500 ng/mL) levels. The human serum pool was also used unspiked. These controls were aliquoted into single use aliquots and stored at −80° C.±15° C. Control stability was assessed during assay validation and was found to be stable after being stored for up to 20 hours 31 minutes at room temperature, up to 40 hours 10 minutes at 2-8° C., and up to 9 freeze-thaw cycles.

Samples were shipped on dry ice and accessioned into Freezer Pro (sample accessioning and tracking software system) and/or Watson LIMS and stored at −80° C.±15° C. Sample stability was assessed during assay validation. Samples were found to be stable after being stored for up to 20 hours 31 minutes at room temperature, up to 40 hours 10 minutes at 2-8° C., and up to 9 freeze-thaw cycles. Prior to use, samples were thawed at room temperature and then subjected to the sample treatment as described further in the assay procedure. For the Titer assay, samples were serially diluted 2-fold in pooled normal human serum and then the appropriate sample dilutions were subjected to the sample treatment as described in the assay procedure.

### ADA Assay Procedure

A Maxisorp® plate was coated with 100 μL/well of 5 μg/mL of test drug in 1×PBS. The plate was sealed and incubated for a minimum of 1 hour at room temperature with shaking (˜600 rpm).

Test samples and controls were diluted 2-fold in 2× Sample Diluent in 0.2 mL PCR tubes or plates. Diluted samples and controls were placed into a thermocycler and heated to approximately 65° C. at the maximum heating rate and remained at approximately 65° C. for 20 seconds, and then cooled to approximately 4° C. at the maximum thermal cycler cooling rate. Once cooled, samples and controls were placed on ice until use. Prior to use, samples and controls were vortexed to mix, spun briefly on a microfuge, and then immediately placed back on ice until ready to be diluted.

Samples and controls were diluted 40-fold (i.e., 1-volume sample and 39-volumes of Blocking Buffer; diluted to a MRD of 80) into Blocking Buffer (5% Blocker A). Diluted Samples and controls (100 μL/well) were transferred to a round bottom polypropylene plate, and 50 μL/well of 0.5 M glycine, pH 2.0 was added to the samples and controls in the plate. Samples and controls were acidified for approximately 15 minutes with shaking (˜600 rpm).

While samples and controls were being acidified, the Maxisorp® plate containing the immobilized test drug was washed three times with approximately 300 μL per well of Wash Buffer (1×PBS containing 0.05% Tween® 20). The plate was tapped on absorbent paper to remove any excess liquid and 20 μL/well of 1 M Tris pH 9.5 was added to the wells.

Acidified samples and controls (100 μL/well) were transferred to the plate containing 1 M Tris. The plate was sealed and incubated at room temperature for approximately 5 minutes with shaking. The plate was then transferred to 2-8° C. and incubated overnight with shaking.

After the overnight incubation, the plate was washed three times with approximately 300 per well of Wash Buffer. The plate was tapped on absorbent paper to remove any excess liquid, and 75 μL/well of 0.5 M glycine, pH 2.0 was added to the plate. The plate was incubated at room temperature for approximately 15 minutes with shaking.

While the overnight plate was incubating in acid, 50 μL/well of 1 M Tris, pH 9.5 was added to a standard MSD® plate. After the acid treatment, 50 μL/well from the overnight acid-treated plate was transferred to the MSD® plate containing Tris. The plate was sealed and was incubated at room temperature for approximately 2 hours with shaking.

The MSD® plate was washed three times with approximately 300 μL per well of Wash Buffer. The plate was tapped on absorbent paper to remove any excess liquid and 250 μL/well of Blocking Buffer was added to the plate. The plate was covered and incubated at room temperature for approximately 1 hour with shaking.

The MSD® plate was washed three times with approximately 300 μL per well of Wash Buffer. The plate was tapped on absorbent paper to remove any excess liquid, and 50 μL/well of detection reagent (0.25 μg/mL Ru-test drug in Blocking Buffer) was added to the plate. For confirmatory assay testing, 50 μL/well of detection reagent (0.25 μg/mL Ru-test drug in Blocking Buffer) and detection reagent and unlabelled detection reagent was added (0.25 μg/mL Ru-test drug and 1 μg/mL test drug in blocking buffer) were added to the plate. The plate was covered with a foil seal and was incubated at room temperature for approximately 1 hour with shaking.

The MSD® plate was washed three times with approximately 300 μL per well of Wash Buffer. The plate was tapped on absorbent paper to remove any excess liquid, and 150 μL per well of 2× Read Buffer T was added to the plate. The plate was read on a Sector Imager 6000 (Meso Scale Discovery®).

### Sample Analysis

Detection of ADA followed a multi-tiered approach comprising a screening assay, a confirmatory assay, and a titer assay.

### Screening Assay (Tier 1)

Samples were run in duplicate and designated “detected” or “not detected” based on the mean normalized value of the sample compared to the normalized value cut point of 1.16 determined during validation. Normalized values were calculated as indicated below. A sample was considered “detected” if the mean normalized value was greater than or equal to the established cut point. Samples that were determined as “detected” were progressed to the confirmatory assay.

$$Normalized Value = \frac{Luminescence of Sample or Control}{Luminescence of Plate Negative Control}$$

Alternatively, the normalized value cut point of 1.16 can be multiplied by the luminescence of the plate negative control to generate the equivalent cut point as a luminescence value. The mean luminescence values of the controls and samples can then be compared to this adjusted cut point.

### Confirmatory Assay (Tier 2)

A confirmatory cut point of 39.4% signal inhibition was established during validation. Samples and controls were evaluated in the presence and absence of 1 μg/mL of unlabelled drug. Samples were considered “confirmed” if their luminescence signal was inhibited by equal to or greater than 39.4% with the addition of unlabelled drug in the detection step. Signal inhibition was calculated according to the equation below:

$$\% inhibition = 1 - ( \frac{Signal of Sample with Drug}{Signal of Sample Alone} ) \times 100$$

### Titration Assay (Tier 3)

Samples that were confirmed positive were subjected to 2-fold serial dilutions until the signal fell below the cut point. The titer cut point was determined using the same normalized value cut point of 1.16. The dilution factor above the dilution at which signal fell below the cut point for the first time was multiplied by the MRD to determine the titer value.

### Acceptance Criteria

### Plate Acceptance Criteria

Each plate for the screening, confirmatory and titer assays contained at least two sets of the high and low quality controls (QCs) in duplicate and at least three sets of the Negative Control (NC) run in duplicate. Up to 40 samples were included on each screening plate, up to 16 samples were included on each confirmatory plate, and up to 5 samples were included on each titer plate. All QCs remained in the correct rank order, i.e., the high was above the low and the low was above the negative. A minimum of 3 out of the 4 positive controls (75%) had Coefficients of Variation (CVs) of less than or equal to 30%. Some outliers in the NC values were identified and were excluded from the NC mean luminescence calculation. The mean of the NC luminescence values was between 48 and 309 having a CV of less than or equal to 30%.

In acceptable confirmatory assay runs, the % inhibition of the QCs remained in the correct rank order, i.e., the high was above the low and the low was above the negative. A minimum of 3 out of the 4 positive controls (75%) had CVs of less than or equal to 30%.

### Sample Acceptance Criteria

Samples were evaluated for the presence of antibodies specific to the test drug, which was a therapeutic antibody. Each sample was evaluated at the MRD and run in duplicate. For the screening assay the mean normalized values for each sample was compared to the normalized value cut point of 1.16.

Alternatively, the normalized value cut point may have been multiplied by the mean luminescence of the plate NC to yield the adjusted cut point as a luminescence value; this luminescence value cut point was then compared to the mean luminescence of each sample.

If the sample was equal to or above the cut point and had a CV of ˜30% the sample was considered “detected” and progressed to the confirmatory assay. If the sample had a CV of >30% and both replicates are above the cut point the sample was considered “detected” and progressed to the confirmatory assay. If the sample had a CV of >30% with one value below and one value above the cut point that sample was retested. Samples that fell below the cut point was considered “not detected” and was excluded from the acceptance criteria of replicate CVs of less than 30%.

For the confirmatory assay, the sample was retested without drug and with drug to ensure specificity the drug. A sample was considered “confirmed” if the uninhibited sample remained above the cut point and if the signal was inhibited by greater than or equal to 39.4% with the addition of drug. If the sample in the absence of drug failed to remain above the cut point, it was reclassified as “not detected.”

Samples that showed less than 39.4% signal inhibition by drug were classified as “not confirmed.” If the CV of the inhibited sample was greater than 30%, the sample was retested in the confirmatory assay. Samples that were confirmed positive were subjected to 2-fold serial dilutions (in pooled human sera) and then diluted to the MRD until the sample fell below the cut point. If the sample CV was greater than 30% for the two dilution factors immediately above the cut point, the titer for that sample was repeated. Sample results less than the cut point were rejected if the CV was greater than 30%. Titer was reported as the MRD multiplied by the highest sample dilution factor above where the signal falls below the cut point for the first time.

### Sample Reanalysis

Sample reanalysis was performed on any run that did not meet the acceptance criteria with respect to the control samples or the sample CV, as applicable. Any additional sample reanalysis was performed, for appropriate bioanalytical reasons.

### Statistical Analysis and Calculations

Assay plates were read on an MSD® Sector Imager 6000. Luminescence data and descriptive statistics such as arithmetic means, normalized values, standard deviations, precision (% CV), and % inhibition were determined using Watson LIMS (ThermoFisher Scientific, version 7.4.2) and/or Gens v2.01.12 software (BioTek Instruments, Winooski, Vt.) and Microsoft Excel 2007, as applicable.

### Reporting of Results

Data for the screening assay was reported as “detected” if the value was at or above the established cut point or “not detected” if the value was below the established cut point of 1.16. During assay validation, the % Inhibition cut point for the anti-assay was determined to be 39.4%. Samples that show inhibition above 39.4% were considered “confirmed” and were progressed to the titer assay. The titer was determined by the last dilution at which the sample remained equal to or above the cut point and was multiplied by the MRD when reported. If there was not enough volume for testing, the sample was to be noted as quantity not sufficient (QNS; i.e., not enough volume to test).

### Example 3

### Exemplary Protocol:

An exemplary protocol for detecting ADAs is provided in Table 4. All of the abbreviations have the same meaning as defined above for Example 2.

TABLE 4
Exemplary protocol for detecting ADAs in an Immunogenicity Assay

| Step | |
|---|---|
| No. | Procedure |
| Day 1 | |
| 1 | Add 100 μL/well of 5 μg/mL of a test drug (in 1× PBS) coating solution to Maxisorp ® plate(s). |
| 2 | Seal plate(s) and incubate for a minimum of 1 hour at room temperature with shaking (setting |
| | 3-4). |
| 3 | Transfer 50 μL of samples and controls to 0.2 mL PCR tubes or plates. Dilute 2-fold by adding |
| | 50 μL of 2× Sample Diluent to all samples and controls. |
| 4 | Place tubes in thermal cycler. Use a program that heats at approximately 65° C. for 20 seconds, |
| | and then cools to 4° C. After the thermal cycler has reached 4° C., remove the tubes/plate and |
| | place on ice. |
| 5 | After samples and controls have cooled, vortex to mix and then perform a brief spin in a |
| | microfuge. Immediately return to ice. |
| 6 | Dilute each sample and control 40-fold (MRD80) by adding 10 μL of sample to 390 μL |
| | Blocking Buffer (Volumes may be adjusted proportionally, if necessary). |
| 7 | Transfer 100 μL/well of diluted samples and controls to round bottom polypropylene plates. |
| 8 | Add 50 μL/well 0.5M glycine, pH 2.0 to polypropylene plate(s). Incubate at room temperature |
| | for approximately 15 minutes with shaking (setting 3-4). |
| 9 | Wash the coated Maxisorp ® plate(s) prepared in Step 1 3× with 300 μL 1× PBST. Tap plate(s) |
| | on absorbent paper to remove excess liquid. |
| 10 | Add 20 μL/well of 1M Tris, pH 9.5 to all wells of the coated & washed Maxisorp ® plate(s). |
| 11 | Transfer 100 μL/well of acidified samples and controls to the corresponding location of the |
| | plate(s) containing 1M Tris, pH 9.5. Seal plate(s) and incubate for 5 minutes at room |
| | temperature with shaking. |
| 12 | Transfer plate(s) to 2° C. to shaker set at ~600 rpm. Incubate overnight. |
| Day 2 | |
| 1 | Wash plate(s) 3 times with 300 μL/well Wash Buffer. Tap plate(s) on absorbent paper to |
| | remove excess liquid. |
| 2 | Add 75 μL/well 0.5M glycine, pH 2.0 to overnight plate(s). Incubate at room temperature for |
| | 15 minutes with shaking. |
| 3 | Add 50 μL/well 1M Tris, pH 9.5 to standard MSD ® plate(s). |
| 4 | Transfer 50 μL/well from acid-treated overnight plate(s) to the corresponding location in the 1M |
| | Tris-containing MSD ® plate(s). |
| 5 | Seal plate(s) and incubate for approximately 2 hours at room temperature with shaking. |
| 6 | Wash plate(s) 3 times with 300 μL/well Wash Buffer. Tap plate(s) on absorbent paper to |
| | remove excess liquid. |
| 7 | Add 250 μL/well of Blocking Buffer to the plate(s). Cover plate(s) and incubate with shaking |
| | at room temperature for ~1 hour. |
| 8 | Wash plate(s) 3 times with 300 μL/well Wash Buffer. Tap plate(s) on absorbent paper to |
| | remove excess liquid. |
| 9 | Add 50 μL/well of detection reagent to the plate(s). Cover plate with a foil seal and incubate |
| | with shaking at room temperature for 1 hour. |
| | **For confirmatory assay testing: Add 50 μL/well of detection reagent and detection reagent + |
| | unlabelled test drug to the plate(s) |
| 10 | Wash plate(s) 3 times with 300 μL/well Wash Buffer. Tap plate(s) on absorbent paper to |
| | remove excess liquid. |
| 11 | Add 150 μL/well 2× Read Buffer T. |
| 12 | Analyze samples. |

1. A method for detecting anti-drug antibodies that are antigenic to a drug in a sample comprising:
(a) obtaining a sample suspected to have one or more anti-drug antibodies;
(b) coating a first substrate with the drug to create an immobilized drug coated substrate;
(c) heating the sample for a time period, wherein the heating of the sample reduces drug target binding to the drug;
(d) contacting the sample of step (c) with the first drug coated substrate of step (b) to form an immobilized complex between the drug coated on the substrate and the anti-drug antibody present in the sample; and
(e) detecting the presence of anti-drug antibodies, if present, with a detection reagent.

2. The method of claim 1, wherein the sample is cooled following the heating step (c).

3. The method of claim 1, wherein the sample is further diluted in an antibody blocking buffer following the heating step (c).

4. The method of claim 3, wherein the sample is diluted to the minimum required dilution, wherein the minimal required dilution is a dilution of the sample that yields a detection signal that is similar to that of the diluent.

5. The method of claim 3, wherein the antibody blocking buffer comprises serum albumin, mammalian serum, bovine serum, calf serum, horse serum, goat serum, rabbit serum, mouse serum, human serum, casein, dried milk, commercial blocking agents, or a combination thereof.

6. The method of claim 1, wherein the sample is treated with an acid or a base for a time period, wherein the acid or base treatment disrupts binding of an anti-drug antibody to a drug prior to the contacting step (d).

7. The method of claim 6, wherein the acid comprises glycine, citrate, maleate, formate, fumarate, acetate, phosphate, carbonate, or HCl or combinations thereof.

8. (canceled)

9. The method of claim 6, wherein the base comprises NaOH, KOH, NH₄OH, tris(hydroxymethyl)aminomethane (Tris base), trimethylamine, or bicarbonate salts or combinations thereof.

10. The method of claim 1, wherein the drug coated substrate is washed and a neutralizing agent is added to the substrate prior to the contacting step (d).

11. The method of claim 10, wherein the neutralizing agent comprises an acidic buffer or a basic buffer.

12. The method of claim 11, wherein the neutralizing buffer is a basic buffer having a pH of about 8 to about 11.

13. The method of claim 1, wherein the anti-drug antibody is disassociated from the immobilized complex on the first substrate and immobilized on a second substrate.

14. The method of claim 13, wherein the disassociation of the immobilized complex comprises further treating the immobilized complex on the first substrate with an acid or a base for a time period, wherein the acid treatment disrupts binding of the anti-drug antibody to the immobilized drug.

15. The method of claim 13, wherein the drug remains immobilized upon the first substrate.

16-17. (canceled)

18. The method of claim 1, wherein the detection reagent comprises the drug conjugated to a detectable label.

19-21. (canceled)

22. The method of claim 1 further comprising titering the anti-drug antibody comprising progressively diluting the sample until the detection falls below a cut point.

23. The method of claim 1, wherein the sample is heated to a temperature within a range comprising: about 40° C. to about 100° C., about 50° C. to about 95° C., about 60° C. to about 95° C., about 60° C. to about 85° C., or about 60° C. to about 75° C.

24. The method of claim 1, wherein the sample is heated for a time period comprising: about 1 second, about 5 seconds, about 10 seconds, about 15 seconds, about 20 seconds, about 40 seconds, about 60 seconds, about 1 minute, about 2 minutes, about 5 minutes, about 10 minutes, about 20 minutes, about 30 minutes, or about 1 hour.

25. The method of claim 1, wherein the first substrate is coated with an excess of the drug compared to an amount of the drug present in the sample.

26-32. (canceled)

33. A method for detecting anti-drug antibodies that are antigenic to a drug in a sample comprising:
(a) obtaining a sample suspected to have one or more anti-drug antibodies;
(b) coating a first substrate with the drug to create an immobilized drug coated substrate;
(c) heating the sample, wherein the heating step reduces drug target binding to the drug;
(d) cooling the sample of step (c);
(e) diluting the sample of step (d) in an antibody blocking buffer;
treating the sample of step (e) with an acid or a base to disassociate any drug and anti-drug antibodies to form a solution of disassociated drug and anti-drug antibody complexes;
(g) contacting the solution of step (f) with the first drug coated substrate of step (b) and incubating the solution with the drug coated substrate for a time period to form an immobilized complex between the drug coated on the substrate and the anti-drug antibody present in the solution;
(h) washing the formed complex on the first substrate with a wash buffer to remove disassociated drug originally present in the sample from the solution;
(i) treating the complex of step (h) with an acid or a base to disassociate the complex to form a second solution of the anti-drug antibody, wherein the acid treatment disrupts binding of the anti-drug antibody to the immobilized drug and wherein the drug remains immobilized upon the first substrate; and contacting the solution with a second substrate;
(j) detecting the presence of anti-drug antibodies, if present, by incubating the second substrate with a detection reagent.

34. The method of claim 33, wherein the method has a sensitivity for detecting levels of anti-drug antibodies before the presence of anti-drug antibodies affects one or more parameters comprising, pharmacokinetic, pharmacodynamic, safety, or efficacy.

35. The method of claim 33, wherein the method has a sensivity in terms of mass of anti-drug antibody detected per mL of sample, wherein the sensitivity comprises a range of between 10 ng/mL to 1,000 ng/mL, 100 ng/mL to 1000 ng/mL, 200 ng/mL to 1000 ng/mL, or 250 ng/mL to 500 ng/mL.

36-46. (canceled)
